(19) United States
(12) Patent Application Publication
(10) Pub. No.: US 2026/0209685 A1
(43) Pub. Date: Jul. 23, 2026

(54) SYSTEM AND METHOD FOR CONTINUALLY GROWING HIGH POPULATIONS OF ACTIVE AND ADAPTED FACULTATIVE ANAEROBES USING ORGANIC WASTES CONTAINING INDIGENOUS MICROBIOLOGY

(71) Applicant: Environmental Bioorganic Sciences Corp., Las Vegas, NV (US)

(72) Inventors: James Rodney Dickerson, Las Vegas, NV (US); Malcolm Burbank, Las Vegas, NV (US); Valerie Vivian Rogers, Las Vegas, NV (US)

(73) Assignee: Environmental Bioorganic Sciences Corp., Las Vegas, NV (US)

(21) Appl. No.: 19/140,508

(22) PCT Filed: Dec. 19, 2023
(86) PCT No.: PCT/US23/84834

§ 371 Date: Jun. 18, 2025

Related U.S. Application Data
(60) Provisional application No. 63/433,576, filed on Dec. 19, 2022.

(51) Int. Cl. C12N 1/20; C12M 1/00; C12M 1/26

(52) U.S. Cl. CPC C12N 1/20; C12M 29/00; C12M 33/00

(57) ABSTRACT

The present invention relates to an apparatus, system, and methods for continually growing a population facultative anaerobes to a threshold volume in a growth vessel, adding growth media to the growth vessel to increase the volume to a heel volume, incubating for a period sufficient for the facultative anaerobes to achieve a cannibalistic state and settle, introducing raw wastewater into the growth vessel without disturbing the settled facultative anaerobes to treat the raw wastewater, and removing a volume of treated wastewater from the growth vessel without disturbing the settled facultative anaerobes to reduce the volume to a heel volume and introducing fresh raw wastewater into the growth vessel once the heel volume is attained.

### CROSS REFERENCE TO RELATED APPLICATIONS

This application claims priority to U.S. provisional application No. 63/433,576 filed Dec. 19, 2022, and titled System And Method For Continually Growing High Populations Of Active And Adapted Facultative Anaerobes Using Organic Wastes Containing Indigenous Microbiology, the contents are all incorporated by reference in its entirety.

### TECHNICAL FIELD

The present invention relates to an apparatus, system, and methods for continually growing high populations of active and adapted facultative anaerobes using organic wastes containing indigenous microbiology.

### BACKGROUND

The traditional means of liquid phase growing microbes with aerobic properties, which includes facultative anaerobes, is to produce them in an aerobic environment to increase the speed of replication in the presence of oxygen and to keep the culture well mixed with the growth substrate material.

Aeration may be achieved by one of several methods that combine oxygen transfer with mixing and are well known.

Anaerobic liquid phase production is similar regarding mixing, but under anaerobic environment.

Separation of the microbes, including spores in the case of spore-forming microbes, from liquid growth media may be accomplished in one of several traditional methods with mechanical means such as special filtration, or simple gravitational settling enhanced with chemistry and/or centrifugation to increase the speed of separation.

The focus has always been upon growing these microbes rapidly, in a factory environment for packaging and distribution, at the lowest possible cost.

Whether producing live microbes or spores in liquid phase, separation of the microbes and/or spores from the liquid to reduce the cost of packaging, storage, and transport after production, requires some type of separation for concentration to minimize transporting unnecessary liquids.

The final concentration of microbes or spores is usually placed in some type of plastic container which, in most cases, is not recycled and creates a problem for end users and is harmful to the environment.

There are methods of growing microbes on site using prepared substrates that may be included with the microbes in solid form or separately in packages. In these cases, while the quantity of packaging is reduced; however, the associated costs to produce populations of microbes required for successful outcomes of the bioaugmentation is limited by several factors, including but not limited to the starting quantity and types of microbes and the availability of growth media.

Preparation of the growth substrate liquid, whether in a factory or on site from an available substrate source involves some means of either removing or inactivating indigenous microbes to allow those being added to grow without competition for available food and nutrients to high populations.

Removal may be accomplished by ultrafiltration or other mechanical means, and deactivation, or sterilization may be done thermally, chemically or by other known, external input means.

Both the preparation of the substrate and maintaining growth conditions are expensive in capital cost, maintenance, and operation.

Therefore, what is needed is a means to produce needed microbes for applications on site to eliminate the costs and plastic pollution associated with production, transportation and application of the quantities needed to accomplish the goals and objectives of bioaugmentation in the applications and with added benefits that cannot otherwise be achieved with microbes grown in a factory environment such as continual adaptation with lowest energy input.

What is also needed is a system and method to produce high populations of certain microbes, like those of genus Bacillus, that do not require oxygen, on site, without the inactivation, death, or removal of indigenous microbes prior to use as substrate for growth of the needed microbes.

### SUMMARY OF THE INVENTION

The present invention relates to a system and method for continually growing high populations of active and adapted facultative anaerobes using organic wastes containing indigenous microbiology.

In aspects, the system and method grow active and adapted facultative anaerobes without first deactivating or removing indigenous microbiology.

In aspects, the method comprises growing a population of facultative anaerobes to a threshold volume in a growth vessel, adding growth media to the growth vessel to increase the volume to a heel volume, incubating for a period sufficient for the facultative anaerobes to achieve a cannibalistic state and settle, introducing raw wastewater into the growth vessel without disturbing the settled facultative anaerobes to treat the raw wastewater, and removing a volume of treated wastewater from the growth vessel without disturbing the settled facultative anaerobes to reduce the volume to a heel volume and introducing fresh raw wastewater into the growth vessel once the heel volume is attained.

In one embodiment, there is provided a method for producing active- and source water-adapted facultative anaerobes in a vessel defining a volume for containing liquid, the method comprising:
seeding, in the vessel, a heel starter culture comprising facultative anaerobes in culture media in a heel starter amount to settle on a base of the vessel;
receiving, in the vessel, source water comprising food source for the facultative anaerobes; directing the source water towards the base of the vessel;
growing the starter culture under anaerobic conditions for a growth period sufficient to induce growth of the facultative anaerobes into a cannibalistic state;
filling the vessel with the source water to produce a mixture comprising the source water and facultative anaerobes until the volume of the mixture is at or slightly below an operating volume of the vessel;
outflowing the mixture out of the vessel through an outflow port emplaced at a height above the base until the volume of the mixture is decreased to a heel volume that is determined by the height of the outflow port above the base;
introducing an amount of spore state culture comprising facultative anaerobes into the mixture at the heel volume; and
harvesting the outflowed mixture for the active- and source water-adapted facultative anaerobes.

In one embodiment, there is provided a vessel for carrying out the method for producing active- and source water-adapted facultative anaerobes, wherein the vessel comprises:
side walls and a base connecting the side walls defining the interior volume for containing an operating volume of liquid;
an opening for receiving a heel starter culture comprising facultative anaerobes in culture media in a starter amount;
a channel for directing source water comprising food source for the facultative anaerobes to the base for receiving the heel starter culture thereon; and
an outflow port emplaced at a height above the base configured to allow outflowing of contents in the vessel through the side walls.

### BRIEF DESCRIPTION OF THE DRAWINGS

FIG. 1 is a schematic showing an apparatus for continually growing high populations of active and adapted facultative anaerobes according to an embodiment of the present invention; and

FIG. 2 is a flowchart of a method for continually growing high populations of active and adapted facultative anaerobes according to an embodiment of the present invention.

### DESCRIPTION OF THE INVENTION

The terminology used herein is for the purpose of describing embodiments only and is not intended to be limiting of the invention.

As used herein, the singular forms “a”, “an” and “the” are intended to include the plural forms as well, unless the context clearly indicates otherwise.

Furthermore, to the extent that the terms “including”, “includes”, “having”, “has”, “with”, or variants thereof are used in either the detailed description and/or the claims, such terms are intended to be inclusive in a manner like the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc. The term “about” or “approximately” means within an acceptable error range for the value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value.

Detailed embodiments of the instant invention are disclosed herein; however, it is to be understood that the disclosed embodiment is merely exemplary of the invention, which may be embodied in various forms and is in no way intended to limit the invention, its application or uses. Therefore, specific composition ranges disclosed herein are not to be interpreted as limiting, but merely as a basis for the claims and as a representation basis for teaching one skilled in the art to variously employ the present invention in virtually any appropriately detailed composition. The embodiments of the invention may be practiced without the theoretical aspects presented. Moreover, the theoretical aspects are presented with the understanding that the Applicant does not seek to be bound by the theory presented. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The novel process described herein utilizes two unique properties of Bacillus and similar bacterial species that do not require oxygen to grow, namely anaerobes and facultative anaerobes.

The first property is rapid sedimentation or separation of the microbes and spores from the liquid. While the rates of sedimentation vary from species to species and properties of the growth media, the rates are significant in traditional methods and vessels used for growth and involving mixing.

What has not been considered, and is novel, is the growth in a vessel where natural, gravity separation is encouraged and desired, without mixing to create the high concentration of active microbes in a settled phase.

The reason for this new approach will become evident from the following discussion that describes the second unique property of the microbes being grown, namely cannibalization.

For the purposes of this discussion, one exemplary facultative anaerobe is from genus Bacillus which are gram positive, naturally occurring, and non-pathogenic microorganism.

Facultative anaerobic bacteria from genus Bacillus will become cannibalistic under anaerobic conditions when the available food and/or nutrients levels are incapable of supporting the continuously multiplying community. As well, these microbes are r-strategists, meaning they grow at rates faster than other microbes, like coliforms, under the same environmental conditions, preventing other microbes from thriving by out-competing them for food and nutrients. These microbes are naturally predatory, and this escalates when food and/or nutrients are in short supply.

As well, one factor in the selection of the facultative anaerobic bacteria is that it can grow at temperatures well below that of ordinary municipal wastewater microbiology. As explained below, this will enhance the overall performance as the microbes are grown at the same temperature as the source water, which is the water to be treated with the microbes being produced.

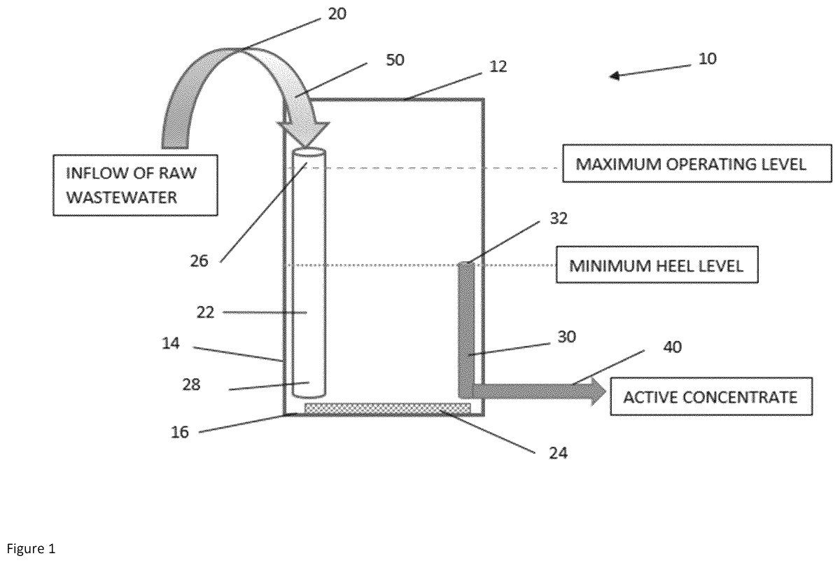

With reference to FIG. 1, there is provided a growth vessel 10 for continually growing high populations of active and adapted facultative anaerobes. The growth vessel 10 leverages the unique properties of using facultative anaerobic bacteria for bioaugmentation and can be any container that ensures a stable source, quantity, and composition of water.

The method and growth vessel 10 attains and maintains a very high concentration of the Bacillus in a settled phase in the bottom of the growth vessel 10, without aeration, such that cannibalistic or near cannibalistic conditions are maintained.

As shown in FIG. 1, the vessel 10 comprises an opening 12, side walls 14, a base 16, and an inflow tube 18 (or a stilling tube) for introducing new water 20 containing food/nutrients and indigenous microbes. As shown, tube 18 defines a channel 22 that delivers new water 20 to the base/bottom 16 of the vessel 10. The delivery is in such a manner as to gently flow new water 18 towards a settled phase 24 containing a very high population of facultative anaerobes (not shown) e.g. active Bacillus, with minimum disturbance of the settled phase 24 of facultative anaerobes at the base 16.

This inflow tube 18 includes an inlet 26 that extends from slightly above the maximum operating level of liquid in the vessel 10 and is dimensioned according to the maximum desired inflow rate to allow the new water 20 to be introduced into the top of the tube 26 to spread out and slow down and gently move down the tube 18 by channeling to the bottom 16 of the vessel 10 before leaving the bottom of the tube 12 though an outlet 28 and entering the settled phase 24 at the base 16 of the vessel 10.

A discharge tube 30, comprising an outflow port 32 is adapted for removing active and adapted Bacillus microbes is placed in the vessel 10 such that an outflow port 32 of the tube 30 would be at or near a minimum heel level (as discussed in detail below) that is maintained with each cycle and inflow into the discharge tube 30 does not entrain a significant portion of the settled phase 24 because the opening is directed upwards towards the surface of the vessel 10 in such a way as to prevent lower levels within the settled phase from leaving the vessel 10.

The rate of inflow of water 20 is adjusted to maintain proper conditions in the settled phase 24 such that indigenous microbes are killed and lysed to provide food/nutrients to the Bacillus population because the population concentration of indigenous microbes is a small fraction of the population of active and aggressive Bacillus within the settled phase 24.

When the liquid in the growth vessel 10 reaches its maximum filled volume, the discharge tube 32 is opened and flow 40 leaves the growth vessel 10 with a large population of active, adapted Bacillus microbes grown using the source water 20 and indigenous microbiology as source of food/nutrients.

The inflow 20 into the stilling tube 18 continues, uninterrupted, as the outflow 40 occurs and when the requisite level in the growth vessel 10 is attained, a small, predetermined quantity of starter culture 50 based upon the volume in the vessel 10 in spore state is introduced into the growth vessel 10.

The purpose of the spore state addition 50 is to introduce a consistent source of genetic material to supplement the adaptation of growing microbes that is occurring within the culture growing in the chamber with each cycle.

In some aspects, maintaining a target operating temperature within the growth vessel 10 will accelerate the multiplication rate of the microbiology in the growth chamber because the replication rate is highly dependent upon the growth chamber temperature. Under preferred conditions, for example, Bacillus can double in population in as little as 20 minutes to as much as 120 minutes, with the ideal range being between 90 F and 103 F.

It is important to note that most common non-Bacillus replication rates are significantly lower than Bacillus, so even if the ideal temperatures are not met for Bacillus growth, most competing indigenous microbiology growth rates will all be significantly lower.

If the population of the Bacillus in the settled phase is significantly higher than the population of microbes entering the growth chamber, the Bacillus will dominate and prevail.

One way to achieve this is, and to ensure this is to create and then maintain a volume of highly concentrated Bacillus before the continual process is begun.

In some aspects, one way to obtain a volume of highly concentrated Bacillus before the continual process is to operate with a heel which is a retained volume of, for example, at 50% of the operating volume of the growth vessel 10. In some aspects, the retained volume (or heel volume) may vary according to site conditions and operating temperatures but will generally be from about 10% to about 50% or from about 5% to about 50% of the total volume/operating volume of the vessel 10.

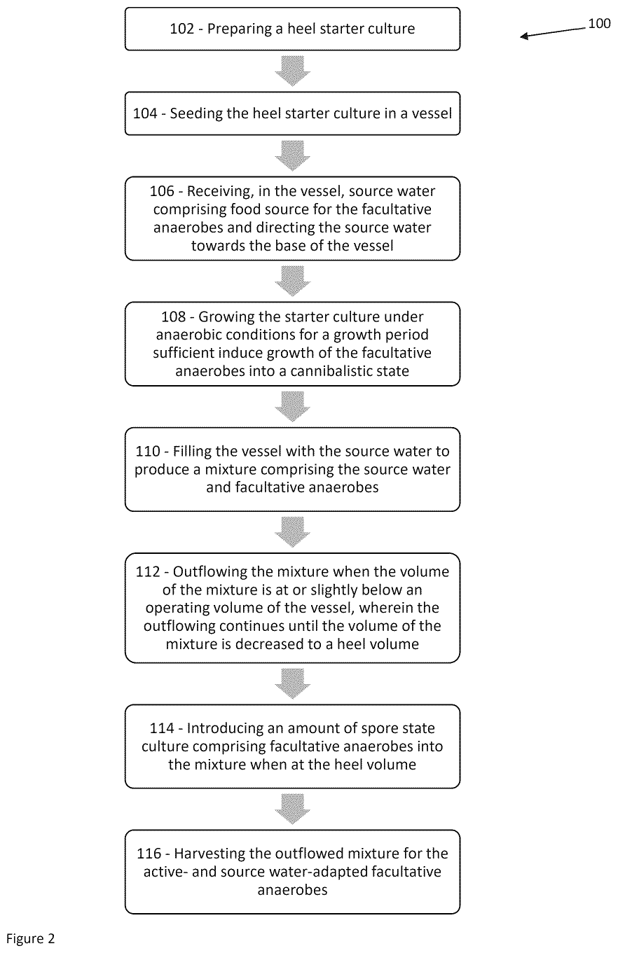

In one embodiment, with reference to FIG. 2, there is provided a process 100 that begins with a preparation stage 102 where there is the preparation of a heel culture using a culture media to activate and grow a volume containing a high population of facultative anaerobes, in this example, a Bacillus formulation. At a seeding stage 104, a heel starter culture comprising facultative anaerobes in culture media in a starter amount is seeded in the vessel. Preferably, in one embodiment, the starting volume of the heel starter culture is from about 5% and about 10% of a heel volume which itself is from about 5% to about 50% of the total volume of the vessel 10. A heel height is the height of the liquid of the heel volume above the base.

Once the heel starting volume is in place in the growth chamber 10 at optimum, at a receiving step 106, a source water 20 such as waste water comprising food source for the facultative anaerobes is introduced into the vessel 10 in an amount to reach the entire heel volume.

This culture media may be synthetic or made from the source water (such as but not limited to wastewater) that will be treated by removing, inactivating, pasteurizing, or sterilizing the source water until the heel volume is achieved.

At a growth step 108, the heel comprising the concentrated facultative anaerobes is then held for sufficient growth period. In aspects, this growth period is the time needed to ensure rapid expansion of the heel starting populations and can be a minimum of about one or more hours, depending on the species of facultative anaerobe and temperature to ensure rapid expansion of the heel starting populations.

After the growth step 108, there is a filling step 110 where additional source water 20 is directed towards the base 16 of the vessel. At the receiving step 106 and filling step 110, the source water 20 is flowed at a flow rate sufficiently low so as to not stimulate significant mixing of the contents in the vessel 10 and/or so as not to stimulate mixing of the seeded starter culture.

For example, the growth rate of the Bacillus, in this example, is doubling every 20 minutes. Two hours of hold time will increase the population by 128 times assuming logarithmic growth rate.

For example, if the growth chamber had a total volume of 105 liters and operated with a 50% heel, starting with 4 liters of initial culture that 4 liters would increase to the equivalent of 512 liters of the initial concentration.

Therefore, if the starting heel were 50% or 52.5 liters then our starting heel will be at or near cannibalistic state.

In one aspect, during the filling step 110, to ensure this cannibalistic state is maintained in the settled phase, the raw wastewater is introduced into the growth chamber at a constant rate over a 2-hour period until the total volume in the growth tank reaches the maximum or the operating volume of the vessel. In another aspect, to ensure this cannibalistic state is maintained in the settled phase, the raw wastewater introduced into the growth chamber and when total volume in the growth tank reaches the maximum or the operating volume of the vessel the contents are held for at 2-hour period.

The low rate of the raw wastewater with indigenous microbes into the proportionately large volume of settled phase will ensure the predatory and cannibalistic nature of the Bacillus in the settled phase remains engaged.

It will not matter if some, or even all the Bacillus microbes in the settled phase go into spore state because the incoming microbial population will be viewed as new food and nutrients and will be killed upon entry into the settled phase.

At an outflowing step 112, when the total volume in the growth tank reaches the maximum or the operating volume of the vessel, the discharge portal 32 will be opened, and the liquid above the portal entrance/discharge portal 32 exits the growth tank.

Once the level in the growth vessel 10 is decreased at the heel volume or at the level of the discharge port 32, at an introducing step 114, a predetermined quantity of spore state culture 50 in an amount based upon the volume in the vessel 10 is introduced into the growth vessel 10.

At a harvesting step 116, the liquid above the discharge portal 32 that was discharged at the outflowing step 112 will have a high population of growing and adapted Bacillus, which may include spores, depending upon growth conditions, that will continue to multiply after exiting the growth tank 10.

The controlled inflow of source water 20 will continue during the discharge to ensure a constant supply of food and nutrients for the settled volume.

The energy input into the process is minimized to that required to supply the raw wastewater and control the processes if the temperature of the wastewater is neglected.

It is important to remember it is not necessary to heat the growth chamber or the incoming raw wastewater if the growth rates are considered in sizing the components of the process because the Bacillus will always grow faster than most indigenous microbes in wastewater under anaerobic conditions at the same temperature.

It is also important to understand that Bacillus dominated cultures settle rapidly, which ensures the high population in the settled phase when the inflow is slow and does not induce significant mixing in the settled phase.

### Example 1

A study was conducted at a site in Marinette, WI using a 4 million gallon biosolids storage tank filled with two-years of biosolids production and approximately 3 feet of supernatant at the top of the settled biosolids.

This tank was chosen to ensure a stable source, quantity and composition of water for the testing.

Part of the adaptation process is governed by the microbes being grown at temperatures near the temperature of the source water where they will be returned for treatment. This avoids thermal shock which inhibits microbial growth.

The inflow into the specially designed tank for growing (grow tank) has a special conduit (stilling tube) inside the grow tank. In one embodiment, the tube has an inner diameter of about 1 inch and a flow velocity of approximately 0.2 inches per second was used inside the stilling tube within the grow tank for the test to prevent the inflow from stirring up the settled solids in the bottom of the tank. In another embodiment. the flow rate is about 500 ml/min. The stilling tube exit is about 1″ from the bottom of the grow tank to prevent the exit velocity from creating significant mixing.

A retained volume called a heel in the grow tank has a vertical conduit which only allows water to be taken down to a specific volume retained volume within the tank, while ensuring the heel in the bottom is not disturbed. The specific volume may vary according to site conditions and operating temperatures but will generally be between 10% to 50% of the total volume of the grow tank.

The continual existence of the heel in the bottom of the grow tank drives the microbial colony in the bottom of the grow tank into and out of cannibalistic state, which also drives the production of spores as well as active microbes and ensures little to no foreign microbes survive. When Bacillus enter cannibalistic state, they produce toxins capable of killing all other microbes, including the Bacillus offspring produced because they cannot stop replicating until a spore is created. Therein is the additional property of the Bacillus which ensures production of only Bacillus.

The grow tank discharges at intervals ideally of two hours or longer, depending upon conditions of the wastewater which include composition and temperature that affect growth rate.

Between about 1 to 4 milliliters, or more, of microbial concentrate varying in concentration of Bacillus spores between 4-11 million cfu is added to the grow tank at the end of the discharge and into the remaining heel. The addition of the concentrate is to maintain a constant source of consistent genetics in the microbial concentrate which consists of the selected microbes in spore state. This ensures that the desired performance continues with the same genetic drivers even as adaptation to site conditions continues. On-site testing, similar to that reported herein, may be performed to inform any adjustments that may be required in the flow rates within the process, including addition rate of concentrate.

As shown in the results tables below, two testing scenarios were performed to verify the performance. One under a relative warm temperature (where the inflow water slightly warmed) and the other with colder, non-warmed, temperature with both using the same source of wastewater. The cooler temperatures were selected because it is well known that microbial growth is significantly slowed with reduced temperatures.

As shown below, the study demonstrated that even at lower growth rates in lower water temperatures, the outcome with is the same with a similar vegetative count to spore count ratios, with colder weather impacting both growth rates similarly.

### Warm Temperature

| | | | | | average | average spore |
|---|---|---|---|---|---|---|
| | | | | Temp | vegetative | count |
| Sample | Location | Time | pH | (C.) | count (cfu/ml) | (cfu/ml) |
| 7 | Inflow | 8:42 | 8.37 | 15.3 | 3,090 | 1,910 |
| | Source | AM | | | | |
| 3 | Grow | 8:40 | 8.1 | 22.2 | 470,740 | 4,260 |
| | Tank Top | AM | | | | |
| 5 | Grow | 9:48 | 8.12 | 23.2 | 35,000 | 210,000 |
| | Tank | AM | | | | |
| | Heel 1 | | | | | |
| 6 | Heel 2 | 10:55 | 8.23 | | 315,500 | 169,500 |
| | (1 hour | AM | | | | |
| | later) | | | | | |
| | 55% | | | | | |
| 8 | Grow | 12:27 | 8.15 | 22 | 337,447 | 2,553 |
| | Tank Top | PM | | | | |
| 11 | Grow | 1:02 | 8.14 | 25 | 108,167 | 116,500 |
| | Tank | PM | | | | |
| | Heel 1 | | | | | |
| 12 | Grow | 2:15 | 8.17 | 19.5 | 940,000 | 1,070,000 |
| | Tank | PM | | | | |
| | Heel 2 | | | | | |

### Cool Temperature

| | | | | | average | average spore |
|---|---|---|---|---|---|---|
| | | | | | vegetative | count |
| Sample | Location | Time | pH | Te(C.) | count (cfu/ml) | (cfu/ml) |
| 14 | Grow | 11:56 | 8.24 | 18.8 | 2,573 | 3,727 |
| | Tank Top | AM | | | | |
| 15 | Heel 1 | 12:00 | 8.17 | 23.4 | 95,667 | 178,667 |
| | | PM | | | | |
| 17 | Heel 2 | 12:56 | 8.22 | 19.3 | 260,000 | 146,000 |
| | | PM | | | | |
| 20 | Grow | 8:40 | 8.35 | 14.7 | 3,137 | 2,350 |
| | Tank Top | AM | | | | |
| 21 | Grow | 9:38 | 8.3 | 15.2 | 39,167 | 24,500 |
| | Tank | AM | | | | |
| | Heel 1 | | | | | |
| 23 | Grow | 10:35 | 8.35 | 14.1 | 207,667 | 140,000 |
| | Tank | AM | | | | |
| | Heel 2 | | | | | |

The study demonstrates that introducing the inflow into the bottom of the vessel in a controlled manner that did not induce significant mixing, that settling, and cannibalization will occur even at lower growth rates regardless of the temperature.

The foregoing process is scalable and the optimum specific ratios, holding times and temperatures will be dependent upon the specific wastewater. These can all be estimated in advance in a laboratory environment.

In summary, the disclosed methodology eliminates the need to manufacture microbiology, like Bacillus, in a factory, and the impacts upon the environment associated with producing, packaging, and transporting for applications, as well as the costs. Additionally, the disclosed system and method allow for continuous growth of high populations of active and adapted facultative anaerobes using organic wastes containing indigenous microbiology and in aspects, the system and method grows active and adapted facultative anaerobes without first deactivating or removing indigenous microbiology.

The embodiments, methods, procedures, and techniques described herein are presently representative of the preferred embodiments, are intended to be exemplary and are not intended as limitations on the scope. Changes therein and other uses will occur to those skilled in the art, which are encompassed within the spirit of the invention and are defined by the scope of the appended claims. Although the invention has been described in connection with specific preferred embodiments, the invention, as claimed, should not be unduly limited to such specific embodiments. Indeed, various modifications of the described modes for carrying out the invention, which are obvious to those skilled in the art, are intended to be within the scope of the claims.

1. A method for producing active- and source water-adapted facultative anaerobes in a vessel defining a volume for containing liquid, the method comprising:
seeding, in the vessel, a heel starter culture including facultative anaerobes in culture media in a heel starter amount to settle on a base of the vessel;
receiving, in the vessel, source water including food source for the facultative anaerobes;
directing the source water towards the base of the vessel;
growing the starter culture under anaerobic conditions for a growth period sufficient to induce growth of the facultative anaerobes into a cannibalistic state;
filling the vessel with the source water to produce a mixture including the source water and facultative anaerobes until the volume of the mixture is at or slightly below an operating volume of the vessel;
outflowing the mixture out of the vessel through an outflow port emplaced at a height above the base until the volume of the mixture is decreased to a heel volume that is determined by the height of the outflow port above the base;
introducing an amount of spore state culture including facultative anaerobes into the mixture at the heel volume; and
harvesting the outflowed mixture for the active- and source water-adapted facultative anaerobes.

2. The method of claim 1 wherein the steps of receiving, in the vessel, source water, directing the source water towards the base of the vessel, growing the starter culture under anaerobic conditions, filling the vessel with the source water to produce a mixture, outflowing the mixture out of the vessel through an outflow port, and introducing an amount of spore state culture are continuously performed.

3. The method of claim 1 wherein the culture media is synthetic media or source water.

4. The method of claim 1 wherein the steps of receiving and/or the filling is at a flow rate sufficiently low so as to not stimulate mixing of the contents in the vessel or so as not to stimulate mixing of the seeded starter culture.

5. The method of claim 1 wherein the source water is substantially at ambient temperature.

6. The method of claim 1 wherein the growth period of the starter culture is about 1 hour or more.

7. The method of claim 1 wherein the vessel includes a downwardly extending tube emplaced within the vessel to direct flow to the base of the vessel.

8. The method of claim 1 wherein the heel volume is from about 5% to about 50% of the operating volume of the vessel.

9. The method of claim 1 wherein the height of the outflow port from the base is configured so that volume remaining after the outflowing is the heel volume.

10. The method of claim 1 wherein the heel starter amount is from about 5% to about 10% of the heel volume.

11. The method of claim 1 wherein the method is carried out in the absence of heating the source water to temperatures sufficient to inactivate microorganisms in the source water.

12. The method of claim 1 wherein the outflowing begins after an incubation period during which the volume of the mixture is at or slightly below an operating volume of the vessel.

13. The method of claim 12 wherein the incubation period is at least about 2 hours.

14. The method of claim 1 wherein the facultative anaerobes are Bacillus.

15. The method of claim 1 wherein the spore state culture includes about 4-11 million cfu of facultative anaerobes in the spore state.

16. A vessel for carrying out the method for producing active- and source water-adapted facultative anaerobes of claim 1, wherein the vessel comprises:
side walls and a base connecting the side walls defining the interior volume for containing an operating volume of liquid;
an opening for receiving a heel starter culture including facultative anaerobes in culture media in a starter amount;
a channel for directing source water including food source for the facultative anaerobes to the base for receiving the heel starter culture thereon; and
an outflow port emplaced at a height above the base configured to allow outflowing of contents in the vessel through the side walls.

17. The vessel of claim 16 where the channel is defined by an elongate a tube having an inlet for receiving the source water and an outlet for directing the source water in the direction of the base to minimize stirring of the liquid.

18. The vessel of claim 16 wherein the height above the base is determined by a heel height when the contents are at about 5% to about 50% of the operating volume of liquid in the vessel.

19. The method of claim 6, wherein the growth period of the starter culture is about 2 hours or more.

20. The method of claim 8, wherein the heel volume is from about 10% to about 50% operating volume of the vessel.
